(19) United States
(12) Patent Application Publication
(10) Pub. No.: US 2006/0210625 A1
(43) Pub. Date: Sep. 21, 2006

(54) Sustained release of positively charged pharmacologically active molecules from a matrix containing polymers with polarized oxygen atoms

(75) Inventor: Argaw Kidane, Montgomery Village, MD (US)

Correspondence Address: HELLER EHRMAN WHITE & MCAULIFFE LLP, 1717 RHODE ISLAND AVE, NW, WASHINGTON, DC, 20036-3001, US

(73) Assignee: Shire Laboratories, Inc., Rockville, MD (US)

(21) Appl. No.: 10/980,820

(22) Filed: Nov. 4, 2004

Related U.S. Application Data
(60) Provisional application No. 60/517,170, filed on Nov. 4, 2003.

(51) Int. Cl. A61K 31/717; A61K 9/20; A61K 9/24

(52) U.S. Cl. 424/464; 424/472; 514/57

(57) ABSTRACT

An oral pharmaceutical composition, comprising one or more positively charged, highly water-soluble pharmaceutically active agents such as trospium chloride, and one or more polymers containing polarized oxygen atoms, whereby the active agent(s) form an ion-dipole interaction with the polymer(s) that may be used for an immediate release system, an extended release system or a delayed release system.

### FIELD OF THE INVENTION

The present invention is directed to matrix-type sustained release pharmaceutical formulations containing positively charged therapeutic molecules. The matrix is composed of polymers with polarized oxygen atoms, such as complex polysaccharides.

### BACKGROUND OF THE INVENTION

Highly water-soluble drugs present a significant challenge in the formulation of sustained release preparations. As soon as the dosage unit comes in contact with water, the highly water-soluble drug dissolves giving a rapid initial release that will slow down slightly, yet continue at reasonably fast rate. This is because highly water soluble molecules in a matrix formula, for instance, act as pore formers, creating channels that significantly increase the surface area of contact between the dosage unit and water.

Sustained release preparations containing highly water-soluble drugs are manufactured in a variety of ways. One common way of providing sustained release of highly water soluble drugs is to use high proportions of waxy or hydrophobic materials in the matrix formula.

Another way is to formulate low dose, highly water soluble drugs with polymers that gel and swell when they come in contact with water. A number of researchers in the field have used cellulosic derivatives, for instance, as polymers that gel in the presence of water.

Other approaches include multi-layered tablets, coated tablets or beads, and osmotic capsules or tablets. These approaches entail complex and multi-step processes that raise the cost of manufacturing significantly.

Charged molecules are highly water-soluble and hence present a challenge in formulating sustained release preparations. There remains a need in the art to develop an efficient and simple way to deliver highly water-soluble compounds in a sustained release manner.

### BRIEF DESCRIPTION OF THE DRAWINGS

FIG. 1 depicts the mean dissolution profiles for trospium chloride from matrix tablets.

FIG. 2 shows the viscosities of a 2% w/w hydroxypropylmethylcellulose (HPMC K4M) solution and a 2% w/w solution of HPMC K4M containing trospium chloride at a concentration of 16.7% w/w.

### DETAILED DESCRIPTION OF THE INVENTION

The present invention provides sustained release preparation that would provide a once-a-day administration utilizing interactions (complexation) between positively charged molecules with hydrophilic, polarized oxygen atom-containing, polymer chains. This invention overcomes the challenge of formulating highly water-soluble drugs into a sustained release form through judicious selection of polymers that exhibit interaction with the charged pharmacologically active molecules.

With the present invention, sustained release profiles are obtained by utilizing a unique interaction between therapeutically active, positively charged molecules and the polarized oxygen atoms in the backbone of hydrophilic polymers. This type of interaction is known in the art as an ion-dipole interaction. The tablets or pellets of the present invention likely operate in a manner similar to simple matrix systems, i.e. by dissolution and diffusion, and so in the present application references to matrix formulations are made, even though these are not typical matrix systems.

With the compositions of the present invention, no coating is necessary to slow the release of highly water-soluble, positively charged molecules. However, it may optionally be applied in order to achieve customized release profiles.

The terms “drug” or “(pharmaceutically or therapeutically) active agent” or simply “active” are used in the present specification and claims to mean any highly water-soluble, positively charged compound that is useful for therapeutic, nutritional, or diagnostic purposes. Further, the terms encompass one or more of such highly water-soluble compounds, or one or more of such compounds in composition with any other active agent(s) regardless of their solubility. Additionally, the present invention is intended as useful for the delivery of such agent(s) to any animal, but preferably mammals, and most preferably humans.

By “highly soluble” is meant that as described in the USP as “very soluble” (less than 1 part solvent per 1 part solute) or “freely soluble” (1-10 parts solvent per 1 part solute).

The present invention is not limited to only certain active agents, but is for example applicable to any highly water-soluble, positively charged compound for which controlled release delivery is desired. Molecules with positive charges include, but are not limited to, quaternary ammonium compounds and salts of basic drugs. Preferred quaternary ammonium compounds are clidinium, glycopyrrolate, and propantheline, which are commonly used for peptic ulcers, and trospium chloride, which is an antispasmodic typically used for urinary incontinence. Most preferred in the formulations of the present invention is trospium chloride.

Polymers whose structure includes polarized oxygen (electronegative) atoms include all cellulosic polymers, alginates, gums such as guar and xanthan gums, polyacrylic acid derivatives such as carbomers, carageenan, povidone and its derivatives such as crospovidone, polyethylene oxides, and polyvinylalcohol. Examples of cellulosic polymers that are suitable for the formulations of the present invention include: hydroxypropylmethylcellulose (HPMC), hydroxypropylcellulose (HPC), hydroxyethylcellulose (HEC), methylcellulose (MC), powdered cellulose, cellulose acetate, sodium carboxymethylcellulose, calcium salt of carboxymethylcellulose, and ethylcellulose. Preferred are the cellulosic compounds, and most preferred is HPMC. The present formulations can use one or more of such polymers in the matrix compositions.

Applicants have discovered that a certain interaction occurs between polarized oxygen atom containing-polymers and positively charged molecules, such as trospium chloride, which is exemplified by the dramatic change in viscosity of, for instance, an HPMC K4M solution when a drug in solution is added to it. An HPMC K4M solution was prepared and diluted to 2% by weight (or “w/w”) with either a trospium chloride solution or water. The viscosity more than doubled in the presence of the drug as compared to an HPMC K4M solution without the drug, which indicates a specific interaction between the charged drug molecules and the HPMC polymer chains. Without being bound to any particular theory, it is believed that this interaction leads to a slowed release of the active agent, and thus for the first time controlled release, once-daily formulations of highly water-soluble compounds can be made with relative ease.

In addition, the compositions of the present invention may contain one or more binders to give the tablets/pellets cohesiveness. Such binders are well known in the art, and include such substances as polyvinyl pyrrolidone, starch, Maltrin, methylcellulose, hydroxypropyl methylcellulose, carboxymethyl cellulose, sucrose solution, dextrose solution, acacia, tragacanth and locust bean gum, which may be applied wet. The binding agent may be present in the composition in an amount of from about 0.2 wt. % to about 20 wt. %, preferably from about 5 wt. % to about 15 wt. %.

Optionally, but preferably, the tablet composition can contain one or more lubricants, which may be added to assure proper tableting. Non-limiting examples of lubricants include magnesium stearate, calcium stearate, zinc stearate, stearic acid, polyethylene glycol, leucine, glyceryl behenate, sodium lauryl sulfate, sodium stearyl fumarate, hydrogenated vegetable oils, and other waxes, including but not limited to, beeswax, carnuba wax, cetyl alcohol, glyceryl stearate, glyceryl palmitate, and stearyl alcohol. The lubricant, when present, is typically in an amount of from about 0.1 wt. % to about 20 wt. % of the composition, preferably from about I to about 10 wt. %, and more preferably about 0.3 to about 3.0 wt. %.

The present invention is preferably formulated into a tablet prepared using methods known in the art, including a wet granulation method and a direct compression method. The oral tablets are prepared using any suitable process known to the art. See, for example, Remington's Pharmaceutical Sciences, 18^{th} Edition, A. Gennaro, Ed., Mack Pub. Co. (Easton, Pa. 1990), Chapters 88-91, the entirety of which is hereby incorporated by reference. Typically, the active ingredient, a positively charged therapeutic compound such as trospium chloride, is mixed with pharmaceutically acceptable excipients (e.g., the binders, lubricants, etc. listed above) and compressed into tablets. Preferably, the dosage form is prepared by a wet granulation technique or a direct compression method to form uniform granulates. Alternatively, the active ingredient(s) can be mixed with the granulate after the granulate is prepared. The moist granulated mass is then dried and sized using a suitable screening device to provide a powder, which can then be filled into capsules or compressed into tablets, caplets, or minitablets, as desired.

The system developed is found to be robust, meaning that the formulations are not very sensitive to slight changes in composition and processing parameters. It has also been found that the amount of polymer(s) in the formulas of the present invention can vary from as low as 30% (w/w) to as high as 65% (w/w) total without affecting the drug release rate. Also, highly water-soluble components, such as citric acid and tartaric acid, can be incorporated into the formula in amounts ranging from 5 to 20% (w/w), again without impacting the drug release rate. Other excipients such as silicified microcrystalline cellulose can also be added to the formula in amounts ranging from 10 to 40% (w/w) without affecting the drug release behavior.

Formulations of this invention can also be made into pelletized forms, which can be filled into capsules or dispensed in sachets for sprinkle application. Each pellet is composed of the drug, the cellulosic polymer(s), and other excipients that aid the processing. Intimate contact between the drug and the cellulosic polymer is essential for the interaction that would result in sustained release of the drug. Pellets can be prepared in one of the many ways that are known by those skilled in the art. These include, for example, extrusion spheronization and roller compaction (slugging). In the extrusion-spheronization technique, drug is mixed with polarized, such as cellulosic, polymers and other excipients. The blend is then granulated in a high shear granulator. The wet mass is then passed through an extruder and spheronized using a spheronizer. The pellets are then dried in an oven or fluid bed processor. The dried pellets are either processed further or encapsulated without further processing.

The present invention also provides a method for treating a mammal with a composition according to the present invention. The method involves orally administering such a composition according to the present invention to a mammal, preferably a human, in need of the therapeutic effects of the active agent. Most preferred is the treatment of a human for urinary incontinence with a once-daily dosage of a composition of the present invention where the active agent is trospium chloride.

A pharmaceutical formulation for the delivery of trospium chloride for the effective treatment of urinary frequency, urgency, nocturia, and urge-incontinence associated with detrusor instability, urge syndrome, and/or detrusor hyperreflexia in a human patient comprising a sustained release composition that provides a sustained release of trospium chloride upon oral administration to said patient; and one or more polymers containing polarized oxygen atoms, whereby the trospium chloride will form an ion-dipole interaction with the polymer(s); wherein the pharmaceutical formulation is sufficient to maintain an effective level of trospium chloride in the patient over the course of at least 12 hours without further administration of trospium chloride . The total dosage of trospium chloride may be about 20 to 70 mg producing in a human patient a plasma concentration versus time curve having an area under the curve of about 30,000 pg/ml*hr to about 80,000 pg/ml*hr. The plasma concentration may have a maximum concentration of about 1.5 ng/ml to about 6.0 ng/ml. The plasma concentration may have a minimum concentration of about 0.5 ng/ml to about 1.5 ng/ml. The maximum concentration of value of the said plasma concentration curve may be reached in about 3 to about 24 hours after oral administration.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention is described below in particularity with the following illustrative examples; however, the scope of the present invention is not intended to be, and shall not be, limited to the exemplified embodiments below.

### EXAMPLES

### Example 1

### Trospium Chloride Sustained Release from a Matrix Tablet

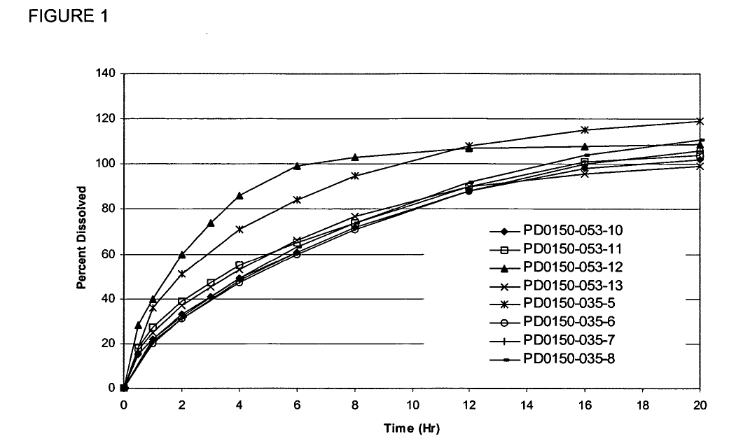

Matrix tablets comprising hydrophilic and hydrophobic polymers were prepared. Trospium HCl was granulated with the polymers using a high shear granulator. Granules were dried in an oven at 40° C. overnight and tableted using a Stokes tablet press. The tablets were evaluated for friability and hardness, as well as tablet weight variation. Table 1 provides the composition of the tablets. FIG. 1 shows dissolution profiles for the tablets.

TABLE 1
Composition of Trospium sustained release matrix tablets

| | Lot # | | | | | | | |
|---|---|---|---|---|---|---|---|---|
| | PD0150- | PD0150- | PD0150- | PD0150- | PD0150- | PD0150- | PD0150- | PD0150- |
| | 035-5 | 035-6 | 035-7 | 035-8 | 053-10 | 053-11 | 053-12 | 053-13 |
| Trospium HCl | 20 | 20 | 20 | 20 | 20 | 20 | 20 | 20 |
| Prosolv HD90 | 10 | 10 | 10 | 10 | 10 | 10 | 40 | 10 |
| Compritol 888ATO | 65.5 | | | | | 20 | 20 | |
| Eudragit S100 | | | | | | 15.5 | 15.5 | |
| Klucel EXF | 4 | 4 | 4 | 4 | 4 | 4 | 4 | 4 |
| Methocel K4M | | 65.5 | 32.75 | 30.25 | 22.75 | 15 | | 22.75 |
| Methocel K100M | | | 32.75 | 30.25 | 22.75 | 15 | | 22.75 |
| Citric acid | | | | 5 | 20 | | | |
| Tartaric acid | | | | | | | | 20 |
| Mag. Stearate | 0.5 | 0.5 | 0.5 | 0.5 | 0.5 | 0.5 | 0.5 | 0.5 |
| Total | 100 | 100 | 100 | 100 | 100 | 100 | 100 | 100 |

### Example 2

### Interaction of Trospium Chloride with HPMC K4M

In order to examine the interaction of trospium chloride with HPMC K4M polymer, viscosity of HPMC K4M was measured at a polymer concentration of 2% w/w at room temperature. Also examined was a 2% w/w solution of HPMC K4M containing trospium chloride at a final concentration of 16.7% w/w.

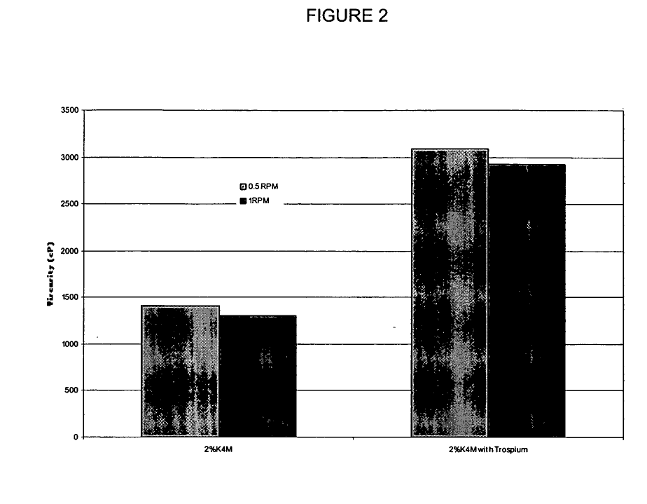

Viscosity was measured using a Brookfield viscometer fitted with an S18 spindle. Viscosity was measured at two spindle speeds. FIG. 2 shows the results. The presence of trospium chloride in the HPMC K4M solution more than doubled the viscosity of the HPMC K4M solution. The contribution of the trospium chloride solution viscosity on the viscosity of the HPMC K4M solution is negligible.

What is claimed is:

1. An oral pharmaceutical composition, comprising one or more positively charged, highly water-soluble pharmaceutically active agents, and one or more polymers containing polarized oxygen atoms, whereby the active agent(s) will form an ion-dipole interaction with the polymer(s).

2. The composition of claim 1, wherein the one or more positively charged, highly water-soluble active agents are quaternary ammonium compounds.

3. The composition of claim 1, wherein the one or more positively charged, highly water-soluble active agents are selected from clidinium, glycopyrrolate, propantheline, or trospium chloride.

4. The composition of claim 3, wherein the active agent is trospium chloride.

5. The composition of claim 1, wherein the one or more polymers containing polarized oxygen atoms are selected from cellulosic polymers, alginates, gums, polyacrylic acid derivatives, povidone and its derivatives, polyethylene oxides, or polyvinylalcohol.

6. The composition of claim 5, wherein the one or more polymers containing polarized oxygen atoms are selected from guar gum, xanthan gum, carbomers, carageenan, or crospovidone.

7. The composition of claim 5, wherein the one or more polymers containing polarized oxygen atoms are selected from hydroxypropylmethylcellulose (HPMC), hydroxypropylcellulose (HPC), hydroxyethylcellulose (HEC), methylcellulose (MC), powdered cellulose, cellulose acetate, sodium carboxymethylcellulose, calcium salt of carboxymethylcellulose, or ethylcellulose.

8. The composition of claim 7, wherein at least one of the polymers containing polarized oxygen atoms is HPMC.

9. The composition of claim 1, which further comprises at least one of a binder and a lubricant.

10. The composition of claim 9, wherein said binder is selected from polyvinyl pyrrolidone, starch, Maltrin, methylcellulose, hydroxypropyl methylcellulose, carboxymethyl cellulose, sucrose solution, dextrose solution, acacia, tragacanth or locust bean gum.

11. The composition of claim 10, wherein the amount of binder present is about 0.2 wt. % to about 20 wt. %, preferably from about 5 wt. % to about 15 wt. %.

12. The composition of claim 11, wherein the amount of binder present is from about 5 wt. % to about 15 wt. %.

13. The composition of claim 9, wherein the lubricant is selected from magnesium stearate, calcium stearate, zinc stearate, stearic acid, polyethylene glycol, leucine, glyceryl behenate, sodium lauryl sulfate, sodium stearyl fumarate, hydrogenated vegetable oils, beeswax, carnuba wax, cetyl alcohol, glyceryl stearate, glyceryl palmitate, or stearyl alcohol.

14. The composition of claim 13, wherein the amount of lubricant present is from about 0.1 wt. % to about 20 wt. %.

15. The composition of claim 14, wherein the amount of lubricant present is from about 1 to about 10% wt. %.

16. The composition of claim 15, wherein the amount of lubricant present from about 0.3 to about 3.0 wt %.

17. The composition of claim 1, which is in the form of a tablet or pellet.

18. The composition of claim 17, wherein the tablet or pellet is surrounded by one or both of an enteric coating and an overcoat.

19. The composition of claim 18, wherein the enteric coating is comprised of one or more enteric materials selected from cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, zein, shellac, copal collophorium, carboxymethyl ethylcellulose, or co-polymerized methacrylic acid/methacrylic acid methyl esters.

20. The composition of claim 19, wherein the enteric coating comprises about 1.0% (w/w) to about 50% (w/w) of the tablet or pellet.

21. The composition of claim 20, wherein the enteric coating comprises about 20 o about 40 percent (w/w) of the tablet or pellet.

22. The composition of claim 18, wherein the overcoat is comprised of a mixture of a sustained release polymer and a water-soluble polymer.

23. The composition of claim 22, wherein the overcoat is comprised of ethylcellulose and hydroxypropylcellulose.

24. The composition of claim 1 wherein the polymer comprises about 30% (w/w) to about 65% (w/w) of the composition.
